(19) United States
(12) Patent Application Publication
(10) Pub. No.: US 2010/0061940 A1
(43) Pub. Date: Mar. 11, 2010

(54) Chewing Gum Compositions Providing Rapid Release of Nicotine

(75) Inventors: Anders Axelsson, Bjarred (SE); Henri Hansson, Helsingborg (SE); Arne Kristensen, Helsingborg (SE)

Correspondence Address: EDWARDS ANGELL PALMER & DODGE LLP, P.O. BOX 55874, BOSTON, MA, 02205, US

(73) Assignee: NicoNovum AB, Helsingborg (SE)

(21) Appl. No.: 12/225,260

(22) PCT Filed: Mar. 16, 2007
(86) PCT No.: PCT/EP2007/002344

§ 371 Date: Mar. 25, 2009

Related U.S. Application Data
(60) Provisional application No. 60/782,903, filed on Mar. 16, 2006.

(30) Foreign Application Priority Data
Mar. 16, 2006 (DK) PA 2006 00375

(51) Int. Cl. A61K 9/68

(52) U.S. Cl. 424/48

(57) ABSTRACT

Use of a nicotine-cellulose combination and a gum base for the preparation of a chewing gum composition for achieving a fast onset of nicotine effect after initiation of chewing the chewing gum composition by a subject. The chewing gum composition is preferably prepared by direct compression and it does not disintegrate during chewing. The invention also relates to chewing gum compositions comprising nicotine, which compositions provide a rapid release of nicotine.

### FIELD OF THE INVENTION

The present invention relates to the use of a nicotine-cellulose combination for the preparation of a chewing gum composition for achieving a fast onset of nicotine effect after initiation of chewing the chewing gum composition by a subject. The invention also relates to chewing gum compositions comprising nicotine, which compositions provide a rapid release of nicotine.

### BACKGROUND OF THE INVENTION

Smoking behavior is associated with serious health risks not only to the smoker, but also to the people around him exposed to passive smoke. To quit smoking has therefore been the expert's advice for many years. However, the smoker is addicted to nicotine, which makes quitting quite difficult for most smokers. Other ways of nicotine administration have been employed in the efforts to help smokers quit their unhealthy habit. Several products employing oral or transdermal administration of nicotine are currently available for smokers wanting to quit smoking, such as chewing gums, inhalators, patches or mouth sprays.

As the tobacco itself contains several other toxic compounds other than nicotine, nicotine substitution products are also relevant for individuals who consume their tobacco in other ways than by smoking. Mainly in Scandinavia, particularly in Sweden, tobacco is consumed as chewing tobacco or snuff. The use of nicotine substitution products will spare consumers of chewing tobacco or snuff as well as smokers from the carcinogenic risks derived from tobacco.

In spite of the availability of several nicotine substitution products such as those mentioned above, many individuals addicted to nicotine still find it difficult to quit their consumption of tobacco. The explanation for this is probably a combination of multiple factors, of which two of them relate to the attained concentration of nicotine in the bloodstream and more importantly, the rate by which nicotine reaches the bloodstream and thereby provides the user with the desired effect.

The rate by which nicotine reaches the bloodstream can be limited by the in vitro rate by which nicotine is released from the nicotine substitution product. Accordingly, there is a need for pharmaceutical compositions comprising nicotine with a rapid release of nicotine, e.g. a rapid in vitro and/or in vivo release. Furthermore, rapid release of nicotine minimizes the total content of nicotine necessary in the compositions, which is a benefit in terms of the consumer's total intake of this potentially toxic compound and in terms of manufacturing economy.

### SUMMARY OF THE INVENTION

The present invention addresses the above-mentioned problems by providing a composition that provides a rapid release of nicotine and a rapid increase in the plasma concentration of nicotine upon in vivo use. The composition may be used as a pharmaceutical composition and/or as a tobacco substitute composition.

Thus, the present invention relates to the use of a nicotine-cellulose combination and a gum base for the preparation of chewing gum composition for achievement of a fast onset of action of nicotine after application of the chewing gum composition to the oral cavity of a subject.

In the present context the term “nicotine-cellulose combination” is intended to denote a solid material composed of a cellulose which has sorbed (adsorbed and/or absorbed) a well-defined amount of nicotine (either as free base or as a pharmaceutically acceptable salt, complex or solvate) e.g. in and/or onto voids or pores within the cellulose. The terms “nicotine-cellulose adduct” and “nicotine-cellulose carrier complex” as used herein are intended to have the same meaning as the term “nicotine-cellulose combination”. As used herein cellulose is an example of a carrier.

A composition of the invention has a fast initial release of nicotine, thus, the composition—when subjected to an in vitro release test—within the first 2 minutes after start of the test releases nicotine with a release rate corresponding to 10% w/w or more of the total content in the composition per minute.

Moreover, a chewing gum composition is non-disintegrating, i.e. it does not disintegrate into particles during chewing of the gum composition, and it does not crumble. It is currently contemplated that use of a particular gum powder as gum base possibly in combination with a suitable selection of additives has impact on the non-disintegrating properties of the chewing gum composition. In specific embodiments, the gum base and/or the chewing gum composition comprises one or more fats, waxes, emulsifiers, plasticizers, oils and/or flavoring agents. Moreover, in a preferred embodiment, the gum base is suitable for direct compression and the chewing gum composition is prepared by direct compression. The chewing gum may be coated or uncoated.

Gum bases having suitable properties and leading to non-disintegrating chewing gum compositions are e.g. gum bases that are or comprise, Gum powder PG 11 TA, Gum powder PG 11 TA New, Gum powder PG 5 TA, Gum powder PG 5 TA New and Gum powder PG N12 TA.

Normally, the gum base is employed in powdered form and has a mean particle size of about 1 mm (as determined by sieving) or less, such as, e.g., about 0.9 mm or less, about 0.8 mm or less, about 0.7 mm or less, about 0.6 mm or less or about 0.5 mm or less.

A fast onset of the nicotine effect is very important in order to be an acceptable product for the consumer. Accordingly, for a chewing gum composition of the invention, the onset takes place within 3 minutes such as, e.g., within 2.5 minutes or within 2 minutes after application of the chewing gum composition to the oral cavity of the subject. In the present context the term “application to the oral cavity” includes initiation of chewing the chewing gum composition.

Accordingly, in another aspect, the invention relates to a composition in solid or semi-solid dosage form, notably a chewing gum composition, comprising nicotine, or a pharmaceutically acceptable salt, solvate, complex, adduct, or derivative thereof, and one or more pharmaceutically acceptable excipients, wherein—when subjected to an in vitro dissolution test as described herein—within the first 2 minutes after start of the test releases nicotine with a release rate corresponding to 10% w/w or more of the total content in the composition per minute.

As it appears from the examples herein, the present inventors have found that compositions in the form of direct compressed chewing gums are especially suitable to achieve a fast release and a subsequent fast appearance of nicotine in the plasma upon in vivo use.

Accordingly, in specific embodiments the invention relates to

i) a direct compressed chewing gum comprising nicotine, or a pharmaceutically acceptable salt, solvate, complex, adduct, or derivative thereof, and one or more pharmaceutically acceptable excipients, wherein—when subjected to an in vitro dissolution test as described herein—within the first 2 minutes after start of the test releases nicotine with a release rate corresponding to 10% w/w or more of the total content in the composition per minute.

Furthermore, the present invention provides methods for preparation of such compositions, comprising mixing nicotine, or a pharmaceutically acceptable salt or derivative thereof, and one or more pharmaceutical acceptable excipients and forming it into a suitable solid or semi-solid dosage form. In one embodiment of the present invention, the dosage form is a chewing gum comprising nicotine, which is obtained by direct compression (DC) of the chewing gum components. The method for preparation of such DC chewing gum comprises mixing the nicotine-containing compound with a gum powder comprising a gum base and one or more pharmaceutical acceptable excipients and compressing this mixture in a tabletting machine.

The present invention also relates to the use of compositions according to the invention, for treatment of nicotine addiction or nicotine withdrawal symptoms.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### DETAILED DESCRIPTION OF THE INVENTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

As mentioned above, the present invention relates to nicotine-containing compositions that release nicotine very fast in order to achieve a very fast rise in plasma concentration upon administration, especially by the oral mucosa. In particular, the invention relates to compositions in a form that is suitable for delivering nicotine to the oral mucosa such as chewing gums.

In a first aspect, the invention relates to a chewing gum composition in solid or semi-solid dosage form comprising nicotine, or a pharmaceutically acceptable salt, solvate, complex, adduct, or derivative thereof, and one or more pharmaceutically acceptable excipients, wherein—when subjected to an in vitro dissolution test as described herein—within the first 2 minutes after start of the test releases nicotine with a release rate corresponding to 10% w/w or more of the total content in the composition per minute. As demonstrated in the examples herein, such a fast release is not obtained by marketed compositions in the form of chewing gum such as Nicorette®. To this end, the present inventors have found that especially directly compressed chewing gum offers advantages over the Nicorette® chewing gum compositions and, furthermore, the use of a nicotine-containing compound in a specific form may also be advantageous in order to obtain as fast a release as possible.

More specifically, the above-mentioned release rate within the first 2 minutes after start of the test is 10% w/w or more such as, e.g., 11% w/w or more, 12% w/w or more, 13% w/w or more, 14% w/w or more or 15% w/w or more of the total content in the composition per minute.

In a specific embodiment a snuff composition according to the invention comprises a carrier comprising internal voids. Such voids may at least partially comprise said nicotine. The carrier is typically insoluble in water or has a low solubility in water. Thus, it typically has a solubility in water at room temperature of less than 1% w/w.

A particular suitable carrier for use in a snuff composition of the invention is a cellulose, such as a microcrystalline cellulose (“mcc”). Certain specific embodiments may also utilize other forms of carriers, in addition to or including mcc, such as but not limited to fibrous material or carbohydrates including cellulose (including hemicellulose, celluloses with different crystallinities and structures (e.g., varying structures including solid fibers, and addition or including fibers or the like in various structures such as web-like structures and/or other structures), including naturally occurring celluloses including Cladophora sp. Algae cellulose or the like), dextran, agarose, agar, pectin, alginate, xanthan, chitosan, starch (including potato starch, shoti starch) etc. or mixtures thereof.

Nicotine may be present in any suitable form such as, e.g. in the form of the free base form of nicotine or in the form of a suitable salt or complex thereof. Moreover, the nicotine may be present in the form of a carrier complex or a carrier adduct, wherein nicotine is present together with a carrier compound. In a specific embodiment, the carrier compound is a particulate material comprising internal voids throughout the material and the voids at least partially comprises said nicotine. While not intended to be bound by theory, it is believed as of the time of this patent application that nicotine may interact with the carrier (for example, mcc or other suitable carrier including other cellulose carriers) by absorbing into and/or adsorbing onto the carrier. Such interaction is completely or nearly completely reversible.

A particular suitable material having internal voids is a cellulose such as, e.g., a microcrystalline cellulose. Specific examples of a suitable microcrystalline cellulose is microcrystalline cellulose selected from the group consisting of AVICEL® grades PH-100, PH-102, PH-103, PH-105, PH-112, PH-113, PH-200, PH-300, PH-302, VIVACEL® grades 101, 102, 12, 20 and EMOCEL® grades 50M and 90M, and the like, and mixtures thereof.

The cellulose may be a synthetic or semi-synthetic cellulose, or it may be derived from natural celluloses.

Suitable carriers may also be those disclosed in WO 2004/064811, which is hereby included by reference.

More specifically, it is contemplated that a relatively high surface area may be of importance for a carrier that is suitable for use. Accordingly, the specific surface area of suitable carriers is normally at least 0.7 m²/g such as, e.g., 1 m²/g. In certain uses, the specific surface area may range between about 0.7 m²/g and at least about 100 m²/g and/or may be anything within this range and/or may be any mixture of sizes within this range. For example, in certain embodiments, the surface area may be about 0.7 m²/g, about 1 m²/g, about 1.5 m²/g, about 2.0 m²/g, about 3.0 m²/g, about 5 m²/g, about 7 m²/g, about 10 m²/g, about 15 m²/g, about 20 m²/g, about 25 m²/g, about 35 m²/g, about 45 m²/g, about 50 m²/g, about 75 m²/g, about 100 m²/g and above about 100 m²/g, or combinations thereof. Such carriers having such suitable surface areas may include, but are not limited to, mcc, fibrous material or carbohydrates including cellulose (including hemicellulose, celluloses with different crystallinities and structures (e.g., varying structures including solid fibers, and addition or including fibers or the like in various structures such as web-like structures and/or other structures), including naturally occurring celluloses including Cladophora sp. Algae cellulose or the like), dextran, agarose, agar, pectin, alginate, xanthan, chitosan, starch (including potato starch, shoti starch) etc. and/or mixtures thereof.

Normally, the mean size range of the carrier compound is from about 15 to about 250 μm.

More specifically, in an embodiment of the invention, nicotine is present as a nicotine-cellulose combination in which said nicotine is at least partly sorbed on cellulose and/or is at least partially absorbed into the carrier and/or is at least partially adsorbed onto the carrier (e.g., mcc), or mixtures thereof. Such interaction is completely or nearly completely reversible.

Hence, in certain specific embodiments nicotine is sorbed on microcrystalline cellulose, absorbed into the mcc and/or adsorbed onto the mcc, and/or combinations thereof.

In embodiments of the present invention, the carrier (e.g., but not limited to mcc and/or other naturally-occurring cellulose) is at least partially porous. This porosity may be due, for example but not limited to, the structure of the carrier, for example, branched, fibrous, or weblike structures may have pores. Ranges of pore sizes include but are not limited to pore volumes of about 0.01 cm³/g and include, but are not necessarily limited to pore volume ranges of from about 0.003 cm³/g or less to about 0.025 cm³/g, to about or greater than 0.60 cm³/g.

In general, the nicotine-cellulose combination is present in a composition of the invention in a concentration of at least about 2% w/w such as in a range from about 2% w/w to about 98% w/w, from about 2% to about 96% w/w, from about 2% w/w to about 95% w/w, from about 3% w/w to about 90% w/w, from about 4% w/w to about 85% w/w, from about 5% w/w to about 80% w/w, from about 5% w/w to about 75% w/w, from about 5% w/w to about 70% w/w, or from about 7.5% w/w to about 65% w/w.

In certain embodiments, the amount of nicotine sorbed, for example absorbed into and/or adsorbed onto to carrier can be up to 50% or more of the total weight of the composition. Ranges of the amount of nicotine sorbed onto the carrier in the present invention range for less than about 1% of the total weight of the composition to more than about 50% of the composition, including all amounts within this range. While applicants do not intend the invention to be bound by theory, it is believed at the time of preparing this application that the maximum amount of nicotine that can be sorbed onto and/or into the carrier, thereby affecting the amount, for example the percent nicotine by weight of the total composition (e.g., the maximum percentage) is affected by properties of the carrier, including but not limited to the structure of the carrier, the porosity of the carrier, and the surface area of the carrier.

In specific embodiments, the concentration of the nicotine-cellulose combination in a composition of the invention is present in a concentration such as, e.g., from about 2% w/w (of the total composition) to about 20% w/w, from about 4% w/w to about 19% w/w, from about 5% w/w to about 18% w/w, from about 6% w/w to about 17% w/w, from about 7% w/w to about 16% w/w or from about 8% w/w to about 15% w/w. In particular this is the case in those situations where the dose required of nicotine is relatively small such as, e.g., in up to a 10 mg range.

In an alternative embodiment, the carrier compound is capable of forming a complex with nicotine such as, e.g., in the case that the carrier compound is an ion-exchange compound including polacrilex.

### Concentrations and Amounts of Nicotine

As mentioned above, nicotine may be present in any suitable form. Normally, nicotine is selected from the group consisting of nicotine base, nicotine hydrochloride, nicotine dihydrochloride, nicotine monotartrate, nicotine bitartrate, nicotine sulfate, nicotine zinc chloride such as nicotine zinc chloride monohydrate and nicotine salicylate. In a preferred aspect, nicotine is in its free base form, which easily can be sorbed on a cellulose to form a microcrystalline cellulose-nicotine carrier complex or carrier adduct.

Normally, the nicotine compound (calculated as the free base) is present in a concentration of at least about 0.1% w/w such as in a range from about 0.1% w/w to about 50% w/w such as, e.g., from about 0.5% w/w to about 45% w/w, from about 1.0% w/w to about 40% w/w, from about 1.5% w/w to about 35% w/w, from about 2% w/w to about 30% w/w, from about 2.5% w/w to about 25% w/w, from about 2.5% w/w to about 20% w/w, from about 3% w/w to about 15% w/w.

Especially in compositions containing a relatively small amount of nicotine (e.g. chewing gums), the concentration of the nicotine compound (calculated as the free base) is normally in a range from about 0.1% w/w to about 15% w/w such as, e.g., from about 0.1% w/w to about 14% w/w, from about 0.1% w/w to about 13% w/w, from about 0.1% w/w to about 12% w/w, from about 0.1% w/w to about 11% w/w, from about 0.1% w/w to about 10% w/w as calculated as free nicotine base.

As mentioned above, the nicotine is present in the form of a nicotine-cellulose combination. In general, this combination is present in a concentration of from about 5% to about 100% such as, e.g., from about 10 to about 100%, from about 5% to about 50% or, alternatively, from about 45% to about 100%. The choice of suitable concentration depends on the load of nicotine in the nicotine-cellulose combination and the dosage of nicotine. If the load is relatively high, then the concentration of the combination may be lower than if the load is relatively low and vice versa. In a specific embodiment using e.g. Avicel® or a similar cellulose quality a concentration of the combination is generally from about 80% w/w to about 98% w/w, such as, e.g., from about 85% w/w to about 98% w/w, from about 90% w/w to about 98% w/w, from about 92% w/w to about 98% w/w, from about 93% w/w to about 97% w/w or from about 94% w/w to about 96% w/w.

The concentration of nicotine (or the pharmaceutically acceptable salt, complex or solvate thereof) in the combination is at the most 70% w/w such as, e.g., at the most 60% w/w, at the most 50% w/w, at the most 45% w/w. The content of nicotine must not be so high that the combination (which is in powder form) “sweats”, so that nicotine desorbs, evaporates or otherwise disappears from the combination. Accordingly, the load of nicotine in the combination is dependent on the particular cellulose employed. If the surface area of the cellulose material is relatively high, then a larger amount of nicotine can be contained therein in a stable manner during a suitable period of time, whereas a cellulose having a smaller surface area normally is indicative for a lower capacity to load nicotine in a suitable manner with respect to stability.

For most cellulose qualities, the concentration of nicotine in the nicotine-cellulose combination is at the most about 45% w/w, such as, e.g., at the most about 40% w/w, at the most about 35% w/w, at the most about 30% w/w, at the most about 25% w/w, at the most about 20% w/w, at the most about 15% w/w, at the most about 12.5% w/w, at the most about 10% w/w, at the most about 9.5% w/w, at the most about 9% w/w, at the most about 8.5% w/w or at the most about 8% w/w, and the concentration being calculated as the nicotine base.

In a specific embodiment, a particulate material according to the present invention has a concentration of nicotine or the pharmaceutically acceptable salt, complex or solvate thereof in the particulate material is at the most about 7.5% w/w such as, e.g., at the most about 7% w/w, at the most about 6.5% w/w, at the most about 6% w/w, at the most about 5.5% w/w, at the most about 5% w/w, at the most about 4.5% w/w, at the most about 4% w/w, at the most about 3% w/w, at the most about 2% w/w or at the most about 1% w/w, and the concentration being calculated as the nicotine base.

The amount of the nicotine compound (calculated as the free base) in a composition of the inventions is generally from about 0.5 mg to about 10 mg such as, e.g., from about 1 mg to about 8 mg, from about 1.5 mg to about 7.5 mg, from about 2 mg to about 5 mg, from about 2.5 mg to about 5 mg, from about 3 to about 10 mg, from about 3 to about 7.5 mg or from about 3 mg to about 5 mg such as, e.g., about 1.5 mg, about 2 mg, about 2.5 mg, about 3 mg, about 3.5 mg, about 4 mg, about 5 mg or about 6 mg, as calculated as free nicotine base. In particular a dosage of 2 mg, 3 mg, 4 mg and 6 mg is of commercial interest.

### Buffering Agents

A composition according to the invention may also contain one or more buffering agents. It is generally known that a slightly alkaline reaction (between 7 and 8) in the oral cavity enhances the absorption of nicotine. Accordingly, it may be and advantage to incorporate a buffer substance in the composition such that a slightly alkaline reaction is provided. Especially compositions for release of the nicotine in the oral cavity can advantageously contain a buffer substance, i.e. compositions like chewing gums, lozenges and snuff compositions.

Suitable buffering agents are typically those selected from the group consisting of acetates, glycinates, phosphates, glycerophosphates, citrates such as citrates of alkaline metals, carbonates, hydrogen carbonates, and borates, and mixtures thereof.

If present the one or more buffering agents are present in a concentration from about 0.5% w/w to about 5% w/w, such as, e.g., from about 0.75% w/w to about 4%, w/w, from about 0.75% w/w to about 3%, w/w or from about 1% w/w to about 2%, w/w.

### Sweeteners

In order to increase the sensory properties of the composition according to the invention one or more sweeteners may be added, such as sugar alcohols including xylitol, sorbitol and/or isomalt, or artificial sweeteners such as e.g. aspartame, acesulfame or saccharin.

The concentration of the one or more sweeteners, if present, is normally at least about 0.05% such as, e.g. from about 0.075% w/w to about 5% w/w or from about 5% to about 35% w/w, such as, e.g., from about 10% w/w to about 35% w/w, from about 15% w/w to about 35% w/w or from about 20% w/w to about 30% w/w.

### Anti-Oxidants

It is well-known that nicotine is subject to oxidation and accordingly, it may be advantageous to incorporate one or more anti-oxidants, such as, e.g., ascorbyl palmitate and/or sodium ascorbate, in a composition according to the invention.

The one or more anti-oxidants may be present in a concentration of from about 0.05% w/w to about 0.3% w/w, such as, e.g., from about 0.1% w/w to about 0.25% w/w or from about 0.15% w/w to about 0.2% w/w.

### Flavouring Agents

In order to improve the organoleptic properties of a composition according to the invention, the composition may include one or more flavouring agents, such as, e.g., menthol flavour, eucalyptus, mint flavour and/or L-menthol, normally present (total concentration of flavouring agents) in a concentration of from about 0.5% w/w to about 12% w/w, from about 1% w/w to about 10% w/w, from about 1.5% w/w to about 9% w/w or from about 2% w/w to about 8% w/w.

### Direct Compressed Chewing Gums (DC Gums)

As mentioned above, an important embodiment of the present invention is a direct compressed (“DC”) chewing gum. As demonstrated in the examples herein, the inventors have found that chewing gums that have been prepared by direct compression have a very favorable rapid initial release of nicotine. The marketed product Nicorette® has not been prepared by direct compression and releases nicotine much slower in the initial phase. Accordingly, the present inventors have found a specific and surprising effect by changing the method for preparing a nicotine-containing chewing gum from the traditionally applied, i.e. mixing of raw materials employing the Bakery type of method followed by extrusion, conditioning, rolling, scoring and finally breaking the gum sheets into individual pieces to direct compression.

Importantly, the present inventors have found that it is crucial to employ specific gum bases in DC compressed nicotine-containing chewing gums in order to obtain a rapid release of nicotine from the composition. Gum bases having properties similar to or substantially similar to the gum bases employed in the examples herein are contemplated as qualities that should be chosen when a chewing gum is prepared by DC due to the fact that such gums have more favorable properties with respect to flowability and compressibility, i.e. properties that are important to enable compression of the gum without e.g. adhesion to the apparatus, incorrect dosing of the gum composition etc.

Direct compressed chewing gum is prepared by using a gum base that is suitable for direct compression together with one or more acceptable excipients normally pharmaceutically acceptable excipients. However, as mentioned above, it is important to use a gum base having suitable properties in order to obtain the desired rapid release. The excipients are selected from the group of excipients normally used within the pharmaceutical industry for the preparation of tablets, i.e. excipients like fillers, disintegrants, binders, lubricants etc. To this end, excipients that enable direct compression are preferred. Guidance may be found in Handbook of Pharmaceutical Excipients edited by Rowe, R. C. et al., 4^{th} edition, Pharmaceutical Press, London 2003, which is hereby incorporated by reference.

Suitable fillers include celluloses and cellulose derivatives including microcrystalline cellulose, hydroxypropylcellulose, sodium carboxymethylcellulose etc.; lactose, starches including potato starch, maize starch etc.

Suitable lubricants include stearates including magnesium stearate, talc, colloidal silica dioxide etc.

Information of the properties of the various marketed gum bases can be obtained from the gum base providers. Suitable gum bases for use in chewing gums according to the invention are obtained in the form of a granular gum base. Specific examples include gum bases provided by e.g. Gumbase Company, Fertin, Gumlink, SPI Pharma, Cafosa, Avant-garde, ATP og Addvantech Pharma and suitable gum bases include Gumpowder PG 11 TA, Gumpowder PG 11 TA New, Gumpowder PG 5 TA, Gumpowder PG 5 TA New and Gumpowder PG N12 TA from Gumbase Company. Other gum bases may be Pharmagum S, Pharmagum M and Pharmagum C from SPI pharma and gum base (Laim J TW A), notably in combination with one or more of the Gumpowders mentioned above. It is important the only gum bases or combinations of gum bases that lead to non-disintegrating chewing gum compositions are employed and, accordingly, the Pharmagum bases may need to be used in combination with other gum bases.

A gum base for use in chewing gums according to the invention is normally in powder or granulate form and has a mean particle size of about 1 mm (as determined by sieving) or less, such as, e.g., about 0.9 mm or less, about 0.8 mm or less, about 0.7 mm or less, about 0.6 mm or less or about 0.5 mm or less.

The gum base is normally present in the chewing gum of the invention in a concentration of from about 25% w/w to about 80% w/w, such as, e.g., from about 30% w/w to about 80% w/w, from about 40% w/w to about 80% w/w or from about 50% w/w to about 80% w/w.

In a chewing gum according to the invention the nicotine is normally present in a concentration from about 0.1% w/w to about 10% w/w such as, e.g., from about 0.1% w/w to about 7.5% w/w, from about 0.1% w/w to about 5% w/w, from about 0.1% w/w to about 2.5% w/w, from about 0.1% w/w to about 1.5% w/w, from about 0.1% w/w to about 1% w/w, from about 0.12% w/w to about 0.8% w/w, from about 0.14% w/w to about 0.6% w/w or from about 0.15% w/w to about 0.4% w/was calculated as free nicotine base.

More specifically, the nicotine is normally present in an amount of from about 0.5 mg to about 10 mg such as, e.g., from about 1 mg to about 8 mg, from about 1.5 mg to about 7.5 mg, from about 2 mg to about 5 mg, from about 2.5 mg to about 5 mg, from about 3 to about 10 mg, from about 3 to about 7.5 mg or from about 3 mg to about 5 mg as calculated as free nicotine base.

In specific embodiments a chewing gum contains 1.5 mg of the nicotine calculated as free nicotine base. The amount 1.5 mg is lower than the marketed Nicorette® chewing gum that contains 2 mg of nicotine. The lowering of the amount of nicotine is due to the observation that a chewing gum according to the invention releases nicotine in such a suitable manner that bioequivalence with respect to AUC is obtained from a 1.5 mg chewing gum when compared with Nicorette® 2 mg. Accordingly, a chewing gum according to the invention has a markedly improved bioavailability of nicotine; in fact the bioavailability is increased by 30%. This, in turn, leads to a reduction in the amount of nicotine in the chewing gum necessary for obtaining the desired effect.

Accordingly, in a separate aspect, the invention relates to a nicotine-containing chewing gum that has a bioavailability that is improved compared with that of Nicorette® and the improvement expressed as the relative bioavailability calculated by AUC_{0-infinity} (tested composition)/AUC_{0-infinity} (Nicorette®)×100% is at least 120% such as, e.g., at least about 130%, at least about 140% or at least about 150%—provided that the composition and Nicorette® contains the same amount of nicotine calculated as free base.

In specific embodiments a chewing gum according to the invention contains 3 mg or 5 mg of said nicotine calculated as free nicotine base.

Nicotine is present in the form of a nicotine-cellulose combination (a carrier complex or a carrier adduct). The carrier complex is typically a nicotine-microcrystalline cellulose carrier complex as described in WO 2004/05663, which is hereby incorporated by reference. Microcrystalline cellulose contains voids that at least partly are filled with the nicotine. One important advantage is that nicotine free base (i.e. in liquid form) easily can fill the voids.

When nicotine is present as a nicotine-microcrystalline cellulose combination and the microcrystalline cellulose has a quality like Avicel® or the like, the concentration of the combination is from about 3% w/w to about 20% w/w, such as, e.g., from about 4% w/w to about 19% w/w, from about 5% w/w to about 18% w/w, from about 6% w/w to about 17% w/w, from about 7% w/w to about 16% w/w or from about 8% w/w to about 15% w/w.

Moreover, the inventors have found that when used in the preparation of direct compressed chewing gum, it is advantageous to employ a quality of microcrystalline cellulose that has a mean particle size that is not too low and neither too high such as, e.g., at the most about 500 μm, at the most about 450 μm, at the most about 300 μm, or at the most about 200 μm, or from about 5 to about 500 μm, from 10 to about 500 μm, from 15 to about 500 μm, from about 20 to about 500 μm, from about 30 to about 500 μm, from about 40 to about 500 μm, from about 10 to about 400 μm, from about 20 to about 400 μm, from about 30 to about 400 μm, from about 40 to about 400 μm, from about 30 to about 300 μm, from about 40 to about 300 μm, from about 50 to about 250 μm, from about 50 to about 200 μm or from about 75 to about 200 μm. In specific embodiments the particle size used were about 100 μm.

As mentioned above, a composition according to the invention may further comprise a pharmaceutically acceptable excipient such as, e.g. a filler, a binder, a lubricant, a buffering agent, a stabilizing agent, a pH adjusting agent, a preservative, a coloring agent, a flavoring agent, a taste-masking agent, a sweetener etc.

In chewing gum composition, a suitable buffering agent is a hydrogen carbonate including alkali metal hydrogen carbonates, or a carbonate including alkaline earth metal carbonates.

If present, sugar alcohols such as, e.g., sorbitol and/or isomalt, may be used in an concentration from about 5% w/w to about 35% w/w, such as, e.g., from about 10% w/w to about 35% w/w, from about 15% w/w to about 35% w/w or from about 20% w/w to about 30% w/w.

As mentioned above, a direct compressed composition according to the invention may further comprise one or more anti-adhesives, lubricants, and/or one or more other pharmaceutically acceptable excipients.

In specific embodiments, the one or more anti-adhesives, lubricants and/or glidants are selected from the group consisting of talc, stearates and salts thereof including magnesium stearate; and silica, and mixtures thereof.

In a specific embodiment, talc is present in a concentration from about 0.5% w/w to about 10% w/w, such as, e.g., from about 1% w/w to about 8% w/w, from about 1.25% w/w to about 6% w/w or from about 1.5% w/w to about 4% w/w, and/or magnesium stearate is present in a concentration from about 0.1% w/w to about 5% w/w, such as, e.g., from about 0.2% w/w to about 4% w/w, from about 0.3% w/w to about 3.5% w/w or from about 0.5% w/w to about 3% w/w, and/or silica is present in a concentration from about 0.1% w/w to about 4% w/w, such as, e.g., from about 0.2% w/w to about 3% w/w, from about 0.3% w/w to about 2% w/w or from about 0.4% w/w to about 1.5% w/w.

In specific embodiments the invention relates to:

A nicotine-containing gum comprising

i) a carrier;
ii) nicotine, or a pharmaceutically acceptable salt, solvate, complex or derivative thereof, wherein the nicotine-containing gum releases at least 7.5% w/w nicotine of the total composition within the first two minutes in the in vitro assay described in Ph. Eur using 20 ml phosphate buffer pH 7.4 and a chewing frequency of 43 cycles per minute in this method.

A nicotine-containing gum comprising

i) a carrier;
ii) nicotine, or a pharmaceutically acceptable salt, solvate, complex or derivative thereof, wherein the nicotine-containing gum releases at least 7.5% w/w nicotine of the total composition within the first two minutes in the in vitro assay described in Ph. Eur using 20 ml phosphate buffer pH 7.4 and a chewing frequency of 43 cycles per minute in this method; and
wherein the nicotine-containing gum is made by direct compression.

A direct compression nicotine-containing gum comprising

i) a carrier;
ii) nicotine, or a pharmaceutically acceptable salt, solvate, complex or derivative thereof, wherein the direct compression nicotine-containing gum releases at least 7.5% w/w nicotine of the total composition within the first two minutes in the in vitro assay described in Ph. Eur using 20 ml phosphate buffer pH 7.4 and a chewing frequency of 43 cycles per minute in this method.

A nicotine-containing gum comprising

i) a carrier;
ii) nicotine, or a pharmaceutically acceptable salt, solvate, complex or derivative thereof, wherein the in vivo uptake by a human, as measured by the content of nicotine in the human's serum, is rapid.

A method of delivering nicotine to an individual comprising the steps of delivering to an individual the nicotine-containing chewing gum as described herein.

A method for making a nicotine-containing gum comprising the steps of:

i) preparing a nicotine-containing composition comprising a carrier and nicotine, or a pharmaceutically acceptable salt, solvate, complex or derivative thereof, wherein the in vivo uptake by a human, as measured by the content of nicotine in the human's serum, is rapid,
ii) directly compressing the nicotine-containing composition to form one or more direct compression gums.

A nicotine-containing chewing gum composition comprising

i) a nicotine-cellulose combination (concentration range: 0.5 to 50% w/w)
ii) a gum base (concentration range: 20-75% w/w)
iii) a buffering agent (concentration range: 0-10% w/w such as 2-6% w/w)
iv) one or more artificial sweeteners (concentration range: 0-2% w/w such as 0.1 to 1% w/w),
v) one or more flavouring agents (concentration range: 0-10% w/w such as 2-8% w/w), and
vi) one or more pharmaceutically acceptable excipients (e.g. fillers such as fillers with sweetening ability like sugar alcohols) (concentration range: 0-80% w/w such as 10-75% w/w, 15-70% w/w, 20-75% w/w or 25-50% w/w) the chewing gum optionally being provided with a coating.

All particulars and details mentioned above relating to the chewing gum aspect in general apply mutatis mutandis to the above mentioned specific embodiments.

### Other Aspects

The invention also relates to a method for the preparation of a composition according to the invention. Specific details can be founds in the examples herein and a person skilled in the art will know how to find guidance e.g. from pharmaceutical handbook of how to select suitable excipient and how to prepare such compositions.

In further aspects, the invention relates to the use of a composition according to the invention as a tobacco substitute or for the alleviation of nicotine withdrawal symptoms.

In another aspect the invention, the compositions of the invention is for pharmaceutical use.

The invention is described in more detail in the following figures and non-limiting examples.

The invention is described in more details in the following figures and non-limiting examples.

### LEGENDS TO THE FIGURES

FIG. 1 shows results from in vitro dissolution testing of chewing gums exemplified in Example 2

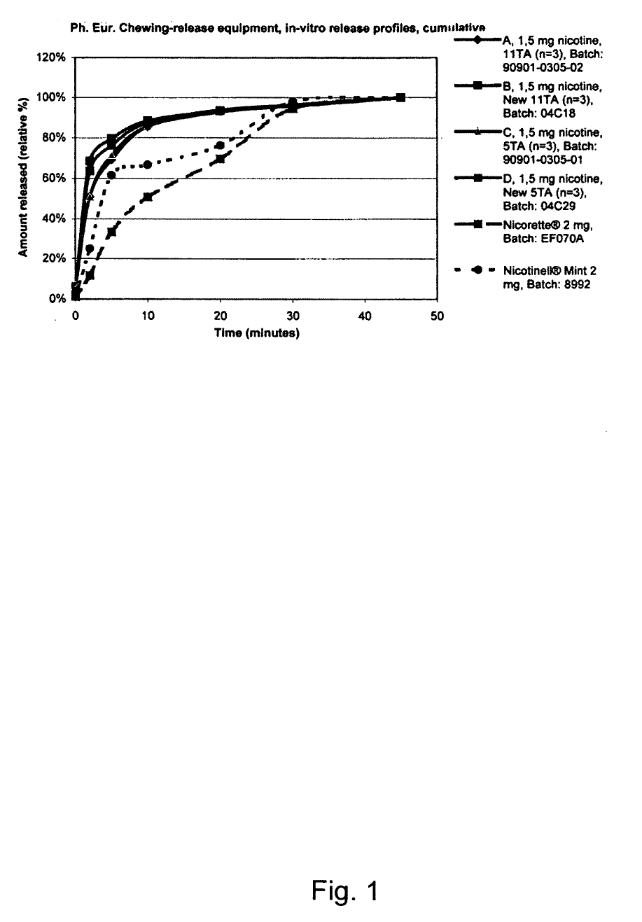

FIG. 2 shows in vivo profiles of DC chewing gum tested as described in Example 2

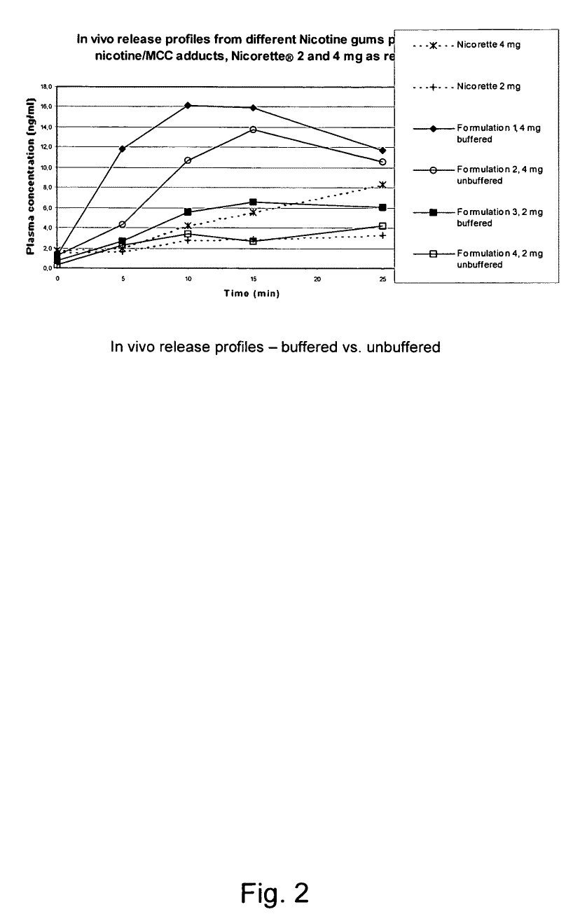

FIG. 3 shows in vivo plasma profiles of a chewing gum according to the invention containing 1.5 mg nicotine and Nicorette® 2 mg

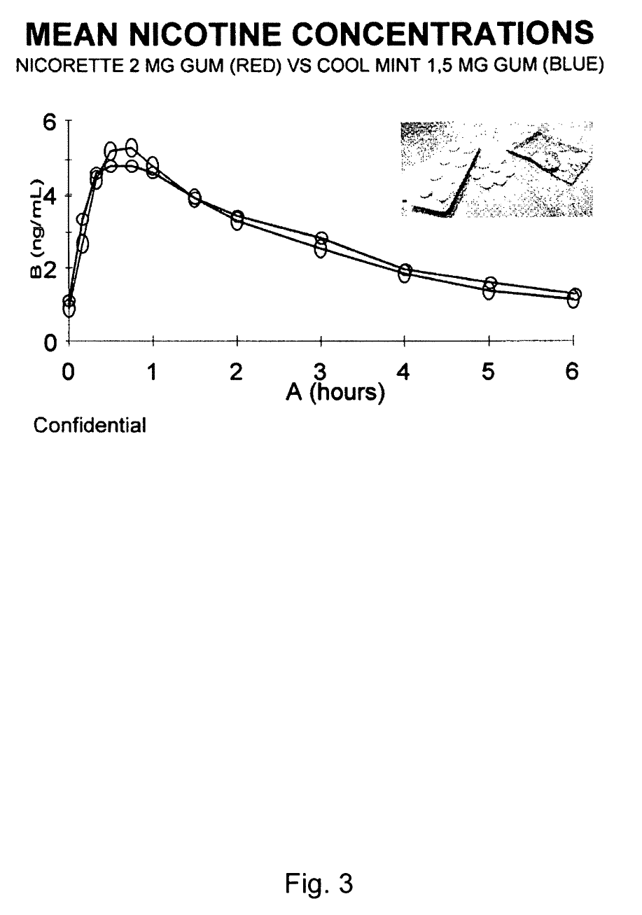

FIG. 4 shows in vivo plasma profiles of buffered or un-buffered DC nicotine chewing gums according to the invention compared with Nicorette® 4 mg

FIG. 5 shows the stability at 30° C. and 65% RH of nicotine DC chewing gums according to the invention (see Example 5 for details)

FIG. 6 shows results of the bioequivalence study in Example 6 with respect to craving

### METHODS

### In Vitro Release Test

The compositions according to the invention must fulfill specific requirements with respect to in vitro release of nicotine. A suitable in vitro test depends on the specific composition in question, i.e. a dissolution test for a chewing gum composition is normally different from a dissolution test for a tablet composition. In general, a person skilled in the art will find guidance as to how to choose a relevant dissolution test for a specific composition in the official monographs such as, e.g., the European Pharmacopoeia. Below is described suitable release or tests in case of chewing gum compositions.

### Chewing Gums

The method and apparatus used were according to Ph. Eur. The chewing apparatus comprises a chewing chamber of 20 mL in which the chewing gum composition is chewed by two horizontal pistons, representing the teeth. The horizontal pistons are capable of rotating around their own axis, which ensures maximum chewing. Together with a third vertical piston (representing the tongue) they work at a constant speed. The pistons are driven by compressed air and their movements are carefully controlled. In more details, the dissolution medium employed was 20 ml phosphate buffer pH=7.4 and a chewing frequency of 43 cycles/min were employed. The dissolution test was run for 45 min. The distance between jaws was 1 mm and the temperature was 37° C.

### EXAMPLES

### Example 1

### Direct Compressed Chewing Gum Compositions A, B, C and D Containing 1.5 Mg of Nicotine

Nicotine was sorbed onto microcrystalline cellulose (MCC) as described in WO 2004/056363. Accordingly, in the present example 2.40 ml nicotine was dissolved in 25 ml ethanol (99.5%). 47.6 g MCC of type PH-102 was loaded into a high-speed mixer and the nicotine was slowly added. After vacuum drying of the obtained wetted mass a fine-grained, white powder of nicotine-microcrystalline cellulose carrier complex was obtained. This was then mixed with the ingredients (except magnesium stearate) stated in the following table in a suitable mixer. Magnesium stearate was sieved and added and the resulting powder mixture compressed into tablets using a tablet press equipped with 17 mm punches. Chewing gum with an average mass of 1.25 g was obtained.

TABLE 1
Gum powder for compositions A, B, C and D

| | A | B | C | D |
|---|---|---|---|---|
| | Concentration | Concentration | Concentration | Concentration |
| Ingredients | (% w/w) | (% w/w) | (% w/w) | (% w/w) |
| Gum powder* from | 39.60 | 39.60 | 40.09 | 39.70 |
| Gumbase | | | | |
| Company | | | | |
| Sorbitol (Ph. Eur. | 23.76 | 24.63 | 24.06 | 24.64 |
| curr. ed.) | | | | |
| Isomalt (Ph. Eur. | 24.50 | 24.60 | 24.88 | 24.60 |
| curr. ed.) | | | | |
| Talc (Ph. Eur. curr. | 3.20 | 3.20 | 3.70 | 3.70 |
| ed.) | | | | |
| Magnesium | 1.50 | 1.50 | 1.70 | 1.70 |
| stearate (Ph. Eur. | | | | |
| curr. ed.) | | | | |
| Silica, colloidal | 0.80 | 0.80 | 0.90 | 0.90 |
| anhydrous (Ph. | | | | |
| Eur. curr. ed.) | | | | |
| Flavours | 6.64 | 5.67 | 4.67 | 4.76 |

*the gum powder employed was for composition A: Gum powder PG 11 TA, for composition B: Gum powder PG 11 TA New, for composition C: Gum powder PG 5 TA and for composition D: Gum powder PG 5 TA New

As flavours may e.g. eucalyptus oil, mint flavour, menthol flavour or the like, and mixtures thereof be used,

### Example 2

### In Vitro Release of Nicotine from Directly Compressed Chewing Gum Compositions

The in vitro release of compositions A, B, C and D prepared as described in Example 1 was investigated and compared with the in vitro release of the marketed products Nicorette® and Nicotinell® both of which containing 2 mg of nicotine.

The in vitro dissolution tests were performed as described above for chewing gums Concentrations of nicotine in the dissolution medium were measured by a HPLC method.

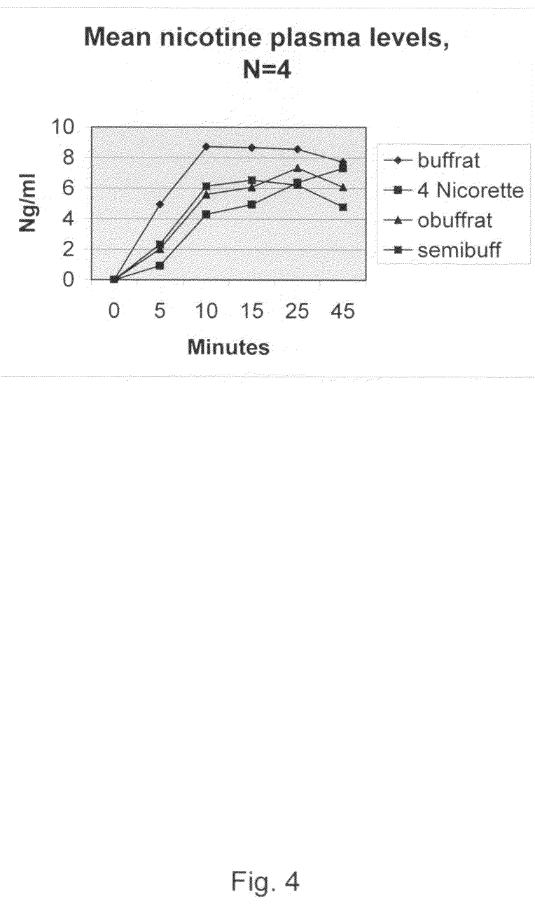

The results are shown in FIG. 4.

Furthermore, the in vitro release of nicotine of composition A was compared to the in vitro release of nicotine of Formula A in WO 00/19977 (Fuisz Technologies Ltd.) and of Nicorette® is shown below:

| | | Composition | Formula A | | |
|---|---|---|---|---|---|
| | Compo- | A- | (WO 00/- | Formula A | |
| | sition | Standardized | 19977) | standardized | Nicorette |
| | A 1.5 mg; | to 2 mg; | 2.2 mg; | to 2 mg; | 2 mg; |
| | nicotine | nicotine | nicotine | nicotine | nicotine |
| Time, | released | released | released | released | released |
| minutes | (μg/min) | (μg/min) | (μg/min) | (μg/min) | (μg/min) |
| 0-2 | 223 | 297 | 120 | 104 | 53 |
| 20-30 | 3 | 4 | | | 25 |
| 3-30 | | | 70 | 63 | |

In conclusion, the present example shows that the directly compressed chewing gum compositions provide a very fast initial release of nicotine in vitro. Furthermore, the initial release is much faster compared to known compositions.

### Example 3

### Buffer Effect on In Vivo Uptake of Nicotine from Dc

In vivo studies have indicated that a faster absorption of nicotine from the oral cavity can be obtained by adjusting the pH of the saliva to pH above 7.

The effect of buffer on the in vivo uptake of nicotine was tested in a comparison study wherein the following formulations were administered to the subject. The formulations 1, 2, 3 and 4 had essentially the same ingredients in the same amounts as that of composition A of Example 1. In order to vary the content of nicotine and to include a buffer substance, the content of isomalt was adjusted accordingly.

Formulation 1: 4 mg nicotine, buffered (10 mg carbonate and 10 mg sodium hydrogen carbonate).

Formulation 2: 4 mg nicotine, unbuffered.

Formulation 3: 2 mg nicotine, buffered (10 mg carbonate and 10 mg sodium hydrogencarbonate).

Formulation 4: 2 mg nicotine, unbuffered.

For comparison, Nicorette® 2 mg and 4 mg chewing gum were included.

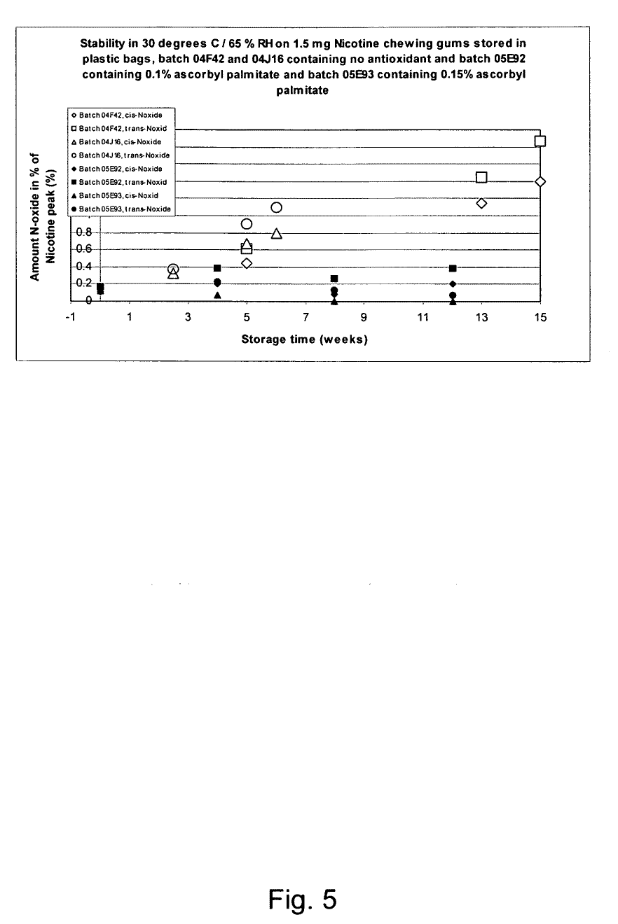

The results are shown in FIG. 5. The results show that the compositions according to the invention have such a fast initial release of nicotine in vitro that even without any buffer substance, they results in in vivo plasma concentrations that are markedly higher than those corresponding to Nicorette® 2 mg or 4 mg, which ever is relevant for comparison purposes. Furthermore, addition of a buffer substance to a composition according to the invention leads to an improved absorption of nicotine. In order words, apart from an initial fast accessibility of nicotine from the compositions according to the invention, a markedly increased absorption of nicotine is seen, i.e. the compositions according to the invention have improved bioavailability (e.g. as measured by AUC or Cₘₐₓ).

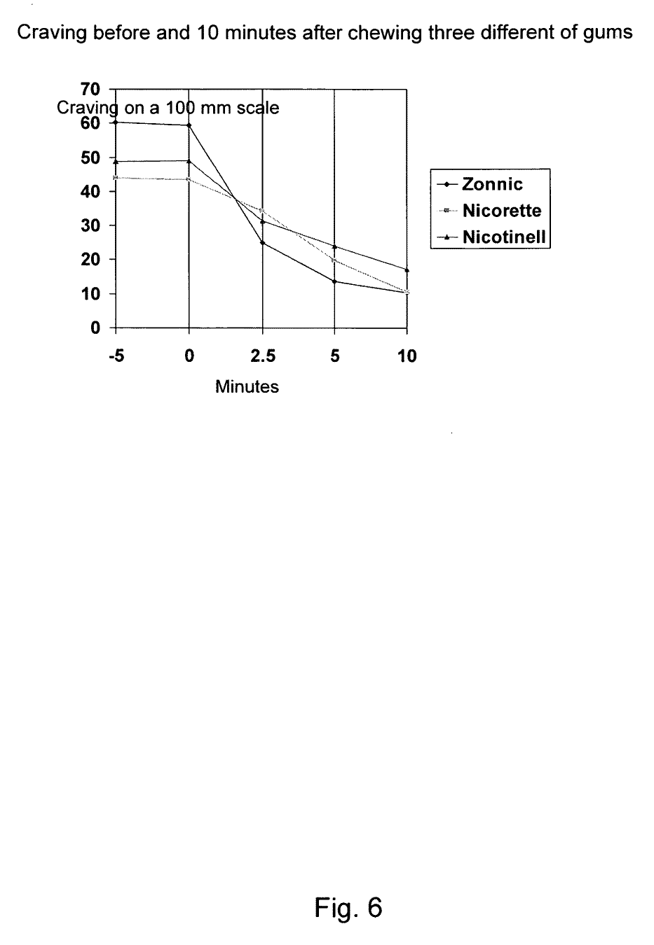

Further studies conducted by the inventors have shown that DC gum without any buffer and containing nicotine in an amount corresponding to 1.5 mg is bioequivalent to Nicorette® chewing gum containing nicotine in an amount corresponding to 2 mg (see FIG. 6.

### Example 4

### DC Gum Compositions Comprising 3 Mg Nicotine

Three different chewing gum compositions containing an amount corresponding to 3 mg nicotine were prepared essentially according to Example 1. One composition was without any buffer substance; another contained a buffer substance (i.e. a mixture of sodium carbonate and sodium hydrogen carbonate). The in vivo uptake was measured (n=4) and the results are shown in FIG. 7. FIG. 7 shows that all DC compositions according to the invention perform better than Nicorette® even if the content of nicotine in the DC compositions according to the invention contain 25% less nicotine than Nicorette®.

### Example 5

### Effect of Antioxidants on the Stability of Nicotine

In order to investigate the effect of anti-oxidants on the stability of nicotine in composition, the amount of the nicotine decomposition products cis-N-oxide and trans-N-oxide was measured for DC gums with containing 0%, 0.1% and 0.15% of the anti-oxidant ascorbyl palmitate, respectively. The level of nicotine decomposition products was measured in the compositions after 2.5, 5, 6, 13, 15 and 16 weeks of storage in plastic bags. The amount nicotine decomposition products were determined by reverse phase HPLC.

The result is shown in FIG. 8 and shows that inclusion of anti-oxidant lowers the decomposition of nicotine in the composition.

### Example 6

### In Vitro Release of Chewing Gum Compositions Comprising a Nicotine-Cellulose Combination and Bioequivalence Study

The following chewing gum compositions were prepared by direct compression essentially as described in Example 1.

The chewing gum composition (A) is coated, medicated chewing-gum containing 3 mg nicotine per unit. It is white to off-white, convex, circular shaped with an approximate total weight of 1.575 g, height of 6.3 mm and diameter of 18.0 mm, depending on the coating. Chewing gum composition (B) contains 1.5 mg nicotine per unit.

### Complete Composition.

| | Comp. A | Comp. B | | |
|---|---|---|---|---|
| | Quantity | Quantity | | |
| Ingredient | (mg/unit) | (mg/unit) | Function | Standard |
| Active substance | | | | |
| Nicotine | 3.30¹ | 1.65¹ | Drug substance | Ph. Eur. curr. |
| | | | | ed. |
| Gum powder PG N12 TA | 926 | | Gum base | Internal, |
| | | | | Gum Base |
| | | | | Co. S.p.A., |
| | | | | Italy |
| Gum powder: PG Nicotine | | 938 | | |
| 5TA/PG New Nik 5TA, Cool mint | | | | |
| flavour | | | | |
| Gum powder: PG Nicotine | | 938 | | |
| 11TA/PG New Nik 11TA, Cool | | | | |
| mint flavour | | | | |
| Microcrystalline cellulose | 121.7 | 60.85 | Nicotine carrier | Ph. Eur. curr. |
| | | | | ed. |
| Isomalt | 120.65 | 241 | Filler, sweetener | Ph. Eur. curr. |
| | | | | ed. |
| Ethanol, anhydrous | 72.0² | 59.75² | Solvent | Ph. Eur. curr. |
| | | | | ed. |
| Ascorbyl palmitate | 2.35 | 2.50 | Antioxidant | Ph. Eur. Curr. |
| | | | | Ed. |
| Acesulfame potassium | 0.500 | 0.500 | Sweetener | Ph. Eur. Curr. |
| | | | | Ed. |
| Aspartame | 0.500 | 0.500 | Sweetener | Ph. Eur. curr. |
| | | | | ed. |
| Silica | | 5.00 | Glidant | Ph. Eur. Curr. |
| | | | | Ed. |
| Core weight | 1175 | | | |
| Coating excipients | | | | |
| Isomalt | 379.5 | 217 | Coating sugar | Ph. Eur. curr. |
| | | | | ed. |
| Purified water | 144.8² | 80.0 | Solvent | Ph. Eur. Curr. |
| | | | | Ed. |
| Ethanol (96 per cent) | 18.0² | 10.9 | Solvent | Ph. Eur. curr. |
| | | | | ed. |
| Acacia | 5.60 | 3.24 | Binder | Ph. Eur. curr. |
| | | | | ed. |
| Titanium dioxide | 5.50 | 3.14 | Colouring agent | Ph. Eur. curr. |
| | | | | ed. |
| Mint liquid flavour | 5.20 | | Flavour | Internal, |
| (O.E. Menta 50/55 | | | | Muller&Koster |
| | | | | S.p.A., Italy |
| Mint liquid flavour | 1.60 | | Flavour | Internal, |
| (Evercool plus Flavour L-124397 | | | | Givaudan |
| | | | | Switzerland |
| | | | | AG |
| Aspartame | 0.271 | 0.155 | Sweetener | Ph. Eur. curr. |
| | | | | ed. |
| Acesulfame potassium | 0.271 | 0.155 | Sweetener | Ph. Eur. Curr. |
| | | | | Ed. |
| Macrogols (Macrogol 6000) | 2.10 | 1.22 | Surface polisher | Ph. Eur. curr. |
| | | | | ed. |
| Coating weight | 400 | 225 | | |
| Total weight | 1575 | 1475 | | |

¹10% overage to compensate for losses during the manufacturing process.
²Evaporates during the manufacturing process.

### Bulk Container and Closure of the Final Product

The coated chewing-gums (final product) are bulk packed in double plastic bags of polyethylene.

The final presentation is in two different packs:

i) aluminium bags, made of Transofoil® LL-OPET/polyethylene; Polyester 12 μm/Aluminium 9 μm/Polyethylene 60 μm containing 20 pieces of chewing gum, and
ii) aluminium blisters, made of PVC/PVDC-foil 250 μm/40 g/m²-20 μm standard aluminium-foil (incl. protective lacquer layer and heat seal lacquer) containing 10 pieces of chewing-gum.

Similar chewing gum compositions but with a content of 1.5 mg of nicotine were tested with respect to in vitro release employing the method described above. Four different gum powders were employed and there were minor variations in the compositions with respect to content of flavours and sweetener. The results were compared with those from Nicorette® 2 mg. The following results were obtained:

A, 1.5 mg nicotine, 11TA (n = 3), Batch: 90901-0305-02

| Time (min) | Cumulative release |
|---|---|
| 0 | 0.064 |
| 2 | 0.509 |
| 5 | 0.691 |
| 10 | 0.855 |
| 20 | 0.936 |
| 30 | 0.964 |
| 45 | 1.000 |

B, 1.5 mg nicotine, New 11TA (n = 3), Batch: 04C18

| Time (min) | Cumulative release |
|---|---|
| 0 | 0.055 |
| 2 | 0.685 |
| 5 | 0.795 |
| 10 | 0.884 |
| 20 | 0.932 |
| 30 | 0.959 |
| 45 | 1.000 |

C, 1.5 mg nicotine, 5TA (n = 3), Batch: 90901-0305-01

| Time (min) | Cumulative release |
|---|---|
| 0 | 0.071 |
| 2 | 0.508 |
| 5 | 0.714 |
| 10 | 0.857 |
| 20 | 0.929 |
| 30 | 0.960 |
| 45 | 1.000 |

D, 1.5 mg nicotine, New 5TA (n = 3), Batch: 04C29

| Time (min) | Cumulative release |
|---|---|
| 0 | 0.063 |
| 2 | 0.636 |
| 5 | 0.762 |
| 10 | 0.874 |
| 20 | 0.937 |
| 30 | 0.958 |
| 45 | 1.000 |

Nicorette ® 2 mg, Batch: EF070A

| Time | Cumulative |
|---|---|
| (min) | release |
| 0 | 0.011 |
| 2 | 0.116 |
| 5 | 0.334 |
| 10 | 0.508 |
| 20 | 0.696 |
| 30 | 0.945 |
| 45 | 1.000 |

Moreover, the nicotine chewing gum composition (3 mg composition as described above; in the figures also denoted Zonnic™ 3 mg)) was compared with Nicorette® 4 mg in a bioequivalence (BE) study.

Moreover, the 1.5 mg composition and Nicorette® 2 mg were subjected to a consumer test carried out in 23 smokers. The results showed that “time to first effect”, i.e. the time it take to sense a nicotine effect after start of chewing, was about 120 seconds for the composition according to the invention, whereas it was 247 seconds for the Nicorette® composition, i.e. a clear indication that a chewing gum composition according to the invention releases nicotine much faster than Nicorette® and, moreover, that a smaller amount is required, i.e. a faster and more efficient release of nicotine from a composition of the present invention.

On a VAS scale (0-100) the subjects rated “craving for a cigarette”. For the composition of the invention (1.5 mg nicotine), the score dropped by 50 points versus 33 for Nicorette® 2 mg from 5 minutes before administration to 10 minutes after administration, which also supports the much faster and more efficient release of nicotine from a composition of the invention compared with Nicorette®:

### REFERENCES

All patents and publications mentioned in the specification are indicative of the levels of those skilled in the art to which the invention pertains. All patents and publications are herein incorporated by reference to the same extent as if each individual publication was specifically and individually indicated to be incorporated by reference.

Although the present invention and its advantages has been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1-58. (canceled)

59. A chewing gum composition comprising a nicotine-cellulose combination and a gum base for achievement of a fast onset of action of nicotine after application of the chewing gum composition to the oral cavity of a subject.

60. The composition of claim 59 wherein the composition—when subjected to an in vitro release test—within the first 2 minutes after start of the test releases nicotine with a release rate corresponding to 10% w/w or more of the total content in the composition per minute.

61. The composition of claim 59 wherein the chewing gum composition is non-disintegrating.

62. The composition of claim 59 wherein the onset takes place within 3 minutes.

63. The composition of claim 59 wherein the application to the oral cavity includes initiation of chewing the chewing gum composition.

64. The composition of claim 59 wherein the gum base comprises one or more fats, waxes, emulsifiers, plasticizers, oils and/or flavoring agents.

65. The composition of claim 59 wherein the gum base is suitable for direct compression.

66. The composition of claim 59 wherein the chewing gum composition is prepared by direct compression.

67. The composition of claim 59 wherein the gum base comprises, Gum powder PG 11 TA, Gum powder PG 11 TA New, Gum powder PG 5 TA, Gum powder PG 5 TA New and Gum powder PG N12 TA.

68. The composition of claim 59 wherein the gum base is employed in powdered form and has a mean particle size of about 1 mm (as determined by sieving) or less.

69. The composition of claim 59 wherein the concentration of the gum base in the chewing gum composition is at the most 80% w/w.

70. The composition of claim 59 wherein the concentration of the gum base in the chewing gum composition is from about 25% w/w to about 80% w/w.

71. The composition of claim 59 wherein said release rate within the first 2 minutes after start of the test is 11% w/w or more of the total content of nicotine in the composition per minute.

72. The composition of claim 59 wherein at least 65% w/w of the total content of nicotine in the composition is released within 5 minutes when subjecting the chewing gum composition to an in vitro release test.

73. The composition of claim 59 wherein at least 75% w/w of the total content of nicotine in the composition is released within 10 minutes when subjecting the chewing gum composition to an in vitro release test.

74. The composition of claim 59 wherein onset of a nicotine effect is at the most 5 minutes after a subject has started chewing of the chewing gum composition.

75. The composition of claim 59 wherein the cellulose of the nicotine-cellulose combination comprises internal voids and/or pores.

76. The composition of claim 75 wherein said voids and/or pores at least partially comprise said nicotine.

77. The composition of claim 75 wherein the cellulose is a cellulose derived from a plant, an algae, a bacterium, a fungi, or combinations thereof.

78. The composition of claim 75 wherein the cellulose has a surface area of at least 0.7 m²/g.

79. The composition of claim 75 wherein the cellulose is a crystalline cellulose including a microcrystalline cellulose.

80. A method of treatment and/or prophylaxis of nicotine addiction comprising administering to a subject in need thereof a composition of claim 59.

81. The method of claim 81 wherein the subject is identified as suffering from or susceptible to nicotine addiction.

82. A method for preparation of a chewing gum composition of claim 59 comprising mixing a nicotine-cellulose combination with one or more pharmaceutical acceptable excipients and forming the resulting mixture it into chewing gum by direct compression.
